(19) United States
(12) Patent Application Publication
(10) Pub. No.: US 2017/0356842 A1
(43) Pub. Date: Dec. 14, 2017

(54) REAL-TIME TRACE GAS SENSOR USING A MULTI-MODE DIODE LASER AND MULTIPLE LINE INTEGRATED CAVITY ENHANCED ABSORPTION SPECTROSCOPY

(71) Applicant: Adelphi University, Garden City, NY (US)

(72) Inventors: Gottipaty Rao, West Hempstead, NY (US); Andreas Karpf, Floral Park, NY (US)

(21) Appl. No.: 15/612,699

(22) Filed: Jun. 2, 2017

Related U.S. Application Data
(60) Provisional application No. 62/347,972, filed on Jun. 9, 2016.

(51) Int. Cl. G01N 21/17; G01N 21/31; H01S 5/40; G01N 33/497

(52) U.S. Cl. CPC G01N 21/1702; G01N 33/497; G01N 21/31; H01S 5/4012; G01N 2021/1704

(57) ABSTRACT

A highly sensitive trace gas sensor based on a Fabry-Perot semiconductor laser and cavity enhanced absorption spectroscopy is designed to be capable of measuring sub-ppb concentrations of trace gases in real time. The broad frequency range of the multi-mode Fabry-Perot semiconductor laser spans a large number of absorption lines of the species of interest enabling multiple line integrated absorption spectroscopy which improves the sensitivity of detection. Additionally, the broad wavelength range of the laser excites a large number of cavity modes simultaneously, thereby reducing the sensor's susceptibility to vibration and thermal fluctuations making it suitable for field based monitoring applications. Using a high finesse optical cavity also enhances the sensitivity of the sensor by providing large path lengths, on the order of kilometers, in a small volume. Relatively high laser power is used to compensate for the low coupling efficiency of a broad linewidth laser to the optical cavity.

### CROSS-REFERENCE TO RELATED APPLICATION

This patent application claims priority to U.S. Provisional Patent Application Ser. No. 62/347,972 filed on Jun. 9, 2016, the entire contents of which are incorporated herein by reference in its entirety.

### FIELD OF THE INVENTION

The present invention relates to highly sensitive trace gas sensors capable of measuring sub-ppb concentrations in real-time. Sensors of this type are useful in the detection of trace gas species in the environment and industrial processes as well as in power plant and automobile emissions. In particular, it may be used to detect pollutants, contaminants and explosive vapors, as well as to indicate the presence of drugs, steroids and molecular biomarkers (of numerous diseases and conditions) in samples of exhaled breath. The invention makes use of a novel, spectroscopic approach which is highly selective to the target gas (i.e., its susceptibility to false readings is minimized), and is capable of recording measurements continuously in real-time. It also uses a novel design that is simple, yet insensitive to vibrations and atmospheric temperature changes, thereby making it well suited for field-based monitoring applications.

### BACKGROUND OF THE INVENTION

Monitoring trace gases in a field environment (which is often prone to vibrations) in real-time is of interest in a wide range of fields, including defense and homeland security, environmental monitoring, and medical diagnostics. These applications require both high sensitivity (because the concentrations of the trace species are often at or below the parts-per-billion (10⁹) level), and high specificity of detection (since the target species will be in the presence of other gases such as water vapor, nitrogen, oxygen, carbon dioxide, ammonia, etc.) Laser-based techniques are well suited for this task because they can achieve high sensitivity (especially when combined with long path length techniques) as well as provide high specificity (by targeting specific absorption features of a desired species).

A variety of spectroscopic techniques have been developed for trace gas detection, each having its own merits and limitations. Commonly employed techniques include:
Absorption spectroscopy using long pass absorption cells such as multipass Herriott cells;
High-finesse optical cavity methods (e.g., Cavity Ringdown Spectroscopy, Cavity Enhanced Absorption Spectroscopy, etc.);
Photo-acoustic and quartz-enhanced photo-acoustic spectroscopy; and
Faraday rotation spectroscopy.

The current status of much of this work has been presented in peer reviewed articles by F. K. Tittel, Y. Bakhirkin, A. Kosterev and G. Wysocki, “Recent Advances in Trace Gas Detection Using Quantum and Interband Cascade Lasers,” Rev. of Laser Eng., vol. 34, pp. 275-282, 2006 (“Tittel”); R. F. Curl, F. Capasso, C. Gmachl, A. A. Kosterev, B. McManus, R. Lewicki, M. Pusharsky, G. Wysocki and F. K. Tittel, “Quantum cascade lasers in chemical physics,” Chem. Phys. Lett., vol. 487, pp. 1-18, 2010 (“Curl”); and G. N. Rao and A. Karpf, “External cavity tunable quantum cascade lasers and their applications to trace gas monitoring,” Appl. Opt., vol. 50, pp. A100-A115, 2011 (“Rao”), each of which is incorporated herein by reference in their entirety.

Techniques employing high-finesse optical cavities are of particular interest because they allow one to achieve very high degrees of sensitivity with a compact experimental cell. See J. J. Scherer and J. B. Paul, “CW Integrated Cavity Output Spectroscopy,” Chem. Phys. Lett., no. 307, pp. 343-349, 1999 (“Scherer”); R. Engeln, G. Berden, R. Peeters and G. Meijer, “Cavity enhanced absorption and cavity enhanced magnetic rotation spectroscopy,” Rev. Sci. Instrum., vol. 69, p. 3763, 1998 (“Engeln”); J. B. Paul, L. Lapson and J. G. Anderson, “Ultrasensitive absorption spectroscopy with a high-finesse optical cavity and off-axis alignment,” Appl. Opt., vol. 40, pp. 4904-4910, 2001 (“Paul”); and G. Berden, R. Peeters and G. Meijer, “Cavity ring-down spectroscopy: Experimental schemes and applications,” Int. Reviews in Physical Chemistry, vol. 19, no. 4, p. 565-607, 2000 (“Berden”), each of which is incorporated herein by reference in their entirety. In particular, these techniques provide long path lengths on the order of several km in a small effective volume. Cavity ring-down spectroscopy (CRDS), for example, enables one to obtain the number density of a species in an absolute scale without the need for secondary calibration standards. See Berden and K. K. Lehmann, G. Berden and R. Engeln, “An Introduction to Cavity Ringdown Spectroscopy,” in Cavity Ringdown Spectroscopy Techniques and Applications, Wiley, 2009, pp. 1-26 (“Lehmann”), which is incorporated herein by reference in its entirety. In CRDS, a laser is coupled to a high-finesse optical cavity. Either a pulsed laser or an interrupted continuous wave (cw) laser beam is used to measure the exponential decay of the light exiting the cavity (cavity ring down time) with and without the gas sample. For example, CRDS has been used to detect low concentrations of NO₂. See N. L. Wagner, W. B. Dubé, R. A. Washenfelder, C. J. Young, I. B. Pollack, T. B. Ryerson and S. S. Brown, “Diode laser-based cavity ring-down instrument for NO₃, N₂O₅, NO, NO₂ and O₃ from aircraft,” Atmos. Meas. Tech., vol. 4, pp. 1227-1240, 2011 (“Wagner”) and H. Fuchs, W. P. Dubé, B. M. Lerner, N. L. Wagner, E. J. Williams and S. S. Brown, “A sensitive and versatile detector for atmospheric NO₂ and NOx based on blue diode laser cavity ring-down spectroscopy,” Environ. Sci. Technol., vol. 43, pp. 7831-7836, 2009 (“Fuchs”), each of which is incorporated herein by reference in their entirety. While, CRDS offers high sensitivity of detection and provides an absolute value of the absorption coefficient, it depends on high-speed detection and triggering electronics to record the short-duration decays. Most implementations of CRDS are susceptible to vibrations, and require additional considerations (e.g., vibration isolation, feedback loops) to minimize the effects of these vibrations. See the Wagner article.

Cavity enhanced absorption spectroscopy (CEAS), removes the need for high-speed detection and electronics by monitoring the transmitted intensity. Sample concentrations are determined by comparing the signal with and without the target species present. As with CRDS, some implementations of CEAS can require very precise alignment, and thus be susceptible to vibration. See, I. Courtillot, J. Morville, V. Motto-Ros and D. Romanini, “Sub-ppb NO₂ detection by optical feedback cavity-enhanced absorption spectroscopy with a blue diode laser,” Appl. Phys B., vol. 85, pp. 407-412, 2006 (“Courtillot”), which is incorporated herein by reference in its entirety. When aligned off-axis, the technique avoids the problems caused by vibration in a relatively simple manner as disclosed by the Paul article. Off Axis CEAS is typically implemented by coupling a tunable, single frequency laser to a high finesse optical cavity. When aligned off-axis, a large number of cavity modes are excited, creating a dense mode structure (i.e., the spacing between the modes is narrower than the laser linewidth). As a result, the laser will always be resonant with some set of cavity modes (regardless of slight changes to the cavity length due to vibrations or small drifts in the laser frequency). If the empty cavity losses are known, the absorption due to the target species may be measured by monitoring the time integrated light intensity that leaks out of the cavity as reported by R. Peeters, G. Berden, A. Olafsson, L. J. J. Larrhoven and G. Meijer, “Cavity enhanced absorption spectroscopy in the 10 micrometer region using a waveguide CO₂ laser,” Chemical Physics Letters, vol. 337, pp. 231-236, 2001 (“Peeters”) which is incorporated herein by reference in its entirety, and Engeln.

There are three drawbacks to CEAS with off-axis alignment: 1) It requires a tunable laser system (which adds complexity, vibration susceptibility and expense); 2) The technique requires large cavity mirrors (to allow multiple reflections within the cavity without causing beam overlap on the mirrors)—this limits the length of the cavity (and thus the sensitivity) in applications requiring small cell volume; 3) The technique results in low coupling efficiency of the laser to the cavity resulting in a weak transmitted signal. This occurs because light is only transmitted when the laser line overlaps a cavity resonance. Cavity resonances result from optical fields entering the cavity at different times and interfering together after different numbers of round trips. The transmitted intensity is described by:

$${I}_{out} ( v ) = \frac{{T}^{2}}{{( 1 - R )}^{2} + 4 R {sin}^{2} ( \frac{2 π nvL}{c} )} {I}_{i n} ( v ) \qquad ( 1 )$$

where Iₒᵤₜ(v) is the transmitted spectral density, Iᵢₙ(v) is the input spectral density, T is the mirror transmissivity, R is the mirror reflectivity, c is the speed of light, n is the index of refraction within the cavity, and L is the cavity length. K. K. Lehmann and D. Romanini, “The superposition principle and cavity ring-down spectroscopy,” J. Chem. Phys., vol. 23, pp. 10263-10277, 1996 (“Lehmann 2”), which is incorporated herein by reference in its entirety. The interference results in transmission resonances at frequencies v_{q}=qc/2 nL (q is a positive integer). The cavity transmission can approach unity (for low loss mirrors 1−R≈T) when a narrow linewidth continuous wave (cw) laser excites a single longitudinal (TEM₀₀) mode in a cavity. This ideal case, however, can be technically challenging as it requires that the laser have a linewidth less than the cavity resonance width (typically ˜10's of kHz), be mode matched with the cavity, and be locked such that it does not drift away from the cavity resonance. D. Romanini, I. Ventrillard, G. Méjean, J. Morville and E. Kerstel, “Introduction to Cavity Enhanced Absorption Spectroscopy,” in Cavity-Enhanced Spectroscopy and Sensing, Berlin, Springer-Verlag, 2014, pp. 1-61 (“Romanini”), which is incorporated herein by reference in its entirety. It should be noted that the transmitted intensity at frequencies between the resonances drops to very low levels because the sine function is not near zero, and thus drops to as little as T²/4. In the case of CEAS, as many cavity modes (i.e., transverse and longitudinal) as possible are intentionally excited. However, the average cavity transmission is significantly reduced from that of the ideal case, and is given by Paul as:

$${I}_{out} = \frac{{I}_{i n} {C}_{P} {T}^{2}}{2 ( 1 - R )} \qquad ( 2 )$$

where C_{P} is the cavity coupling parameter (a measure of the spatial mode quality of the beam and the degree of mode matching between the laser and the cavity). It should be noted that Eq. (2) pertains to light transmitted through the rear mirror, and that the factor of ½ comes from the fact that light exits through both front and back cavity mirrors. The cavity coupling parameter will have a value between 0 and 1: C_{P} will approach 1 for a TEM₀₀ cw laser with a high degree of mode matching with the cavity; it will be significantly lower (C_{P}˜0.1) for a pulsed laser. See the Paul article. Thus, exciting a large number of modes allows one to record spectra without gaps caused by the transmission spectrum of the cavity, but the transmitted intensity will be reduced by more than a factor of the mirror transmissivity T from the ideal case. For typical cavity mirrors (R˜0.9998 and T˜0.00005), the cavity transmission may be reduced from the ideal case by more than a factor of 10⁶.

Incoherent Broad Band Cavity Enhanced Spectroscopy (IBB-CEAS) simplifies the apparatus by removing the need for a tunable laser source, while also reducing the sensor's susceptibility to vibration. In this technique, a light emitting diode (LED) is coupled to a high finesse optical cavity to perform trace gas detection. See S. Fiedler, A. Hese and U. Heitmann, “Influence of the cavity parameters on the output intensity in incoherent broadband cavity-enhanced absorption spectroscopy,” Rev. Sci. Instrum., vol. 78, p. 073104, July 2007 (“Fiedler”), which is incorporated herein by reference in its entirety. The broad bandwidth (FWHM is typically 10 to 20 nm) and the diverging nature of light from an LED excites a vast number of longitudinal and transverse cavity modes (over a frequency range of thousands of free spectral ranges (FSR). This provides two benefits: 1) Changes in the cavity length (due to vibration) will not affect the cavity's transmission spectrum since it effectively excites a continuum of cavity modes; 2) The broad bandwidth removes the need to tune the laser. There are two main drawbacks to the IBB-CEAS approach. First, the low coupling efficiency described for CEAS above is exacerbated by the spatial incoherence of the LED's light. As a result, one is only able to couple a fraction of the LED radiation into the entrance aperture of the cavity. The second drawback is that the LED's broad spectrum will reduce the device's specificity (the width of the LED spectrum will likely cover the lines from several species in ambient air). As a result, implementations of the technique require the use of a spectrometer to selectively monitor the absorption of specific lines from the target species. See the articles by Fiedler and Triki. Due to the use of a spectrometer and the weak transmitted signal, IBB-CEAS implementations are typically 10-100 times less sensitive than laser-based CRDS or CEAS, and have response times of minutes rather than seconds as described in the article by Triki.

The use of a multi-mode semiconductor laser with CEAS simplifies the apparatus even further. Laser sources of this type emit dozens of modes, typically in a Gaussian-like envelope with a width on the order of 1 nm. This frequency spread is narrow enough that individual target species can be selectively monitored without the need for a spectrometer (as in IBB-CEAS), but still broad enough that it will excite a large number of cavity modes and remove the need for tuning. As a result, the technique may be implemented using a basic Fabry-Perot semi-conductor laser, high-finesse optical cavity and photodiode. The coupling of LEDs and multi-mode semiconductor lasers to optical cavities has a common problem: Low coupling efficiency. As discussed above, this is primarily due to the low transmission of light by the cell at frequencies between the cavity resonances. Semiconductor lasers have an advantage over LEDs in that the spatially coherent output from a laser can be easily collimated. Thus unlike LEDs, nearly all of a laser's output can be directed into a cavity. In addition, the availability of relatively inexpensive, high power semiconductor lasers further alleviates the problem of low throughput.

The concentration of the species may be obtained using Beer's law:

I(v)=I(v)e^{-α(v)L} (3)

where I(v) is the intensity of light transmitted through the empty cavity, I′(v) is the transmitted intensity with the target species present, L is the optical path length, and a(v) is the absorption coefficient at frequency v. See, J. M. Hollas, High Resolution Spectroscopy, Second Edition, Wiley, 1998 (“Hollas”), which is incorporated herein by reference in its entirety. In the limit of low absorption, the exponential may be expanded to obtain (to first order):

I(v)=I(v)(1−αL) (4)

Beer's law does not generally apply when using a high-finesse cavity. See M. Triki, P. Cermak, G. Mejean and D. Romanini, “Cavity-enhanced absorption spectroscopy with a red LED source for NOx trace analysis,” Appl. Phys. B., vol. 91, p. 195-201, 2008 (“Triki”), which is incorporated herein by reference in its entirety. Specifically, when using a broadband source, the cavity mode structure must be taken into account as well as changes in the reflectivity of the mirrors as a function of wavelength. In the low absorption limit, the transmitted cavity intensity may be written as:

$${T}^{′} = \frac{{T}^{2}}{2 ( 1 - R )} ( 1 - \frac{F}{π} α L ) \qquad ( 5 )$$

where F=ππR^{1/2}/(1−R) is the finesse of the optical cavity. In the case of a multi-mode semiconductor laser, however, the wavelength range of the laser output is narrow enough (˜1 nm), that one can treat R as a constant. See the Triki article. In this case, if T²/[2(1−R)] is taken to be the intensity of the transmitted light without the sample present, then Eq. (5) is equivalent to Beer's law expanded to 1^{st} order with L_{eff}=LF/π. Thus, in the low absorption limit the cavity provides a linear absorption signal gain. See the Paul article.

Multiple line integrated absorption spectroscopy (MLIAS) is a technique where one scans a laser over a large number of ro-vibrational or rovibronic transitions and takes the sum of the areas of all of the absorption peaks (after subtracting the background) for sensitivity measurements. It has been shown that integrating over multiple absorption lines can enhance the sensitivity of detection by over one order of magnitude for species with dense poorly resolved spectra. See A. Karpf and G. N. Rao, “Enhanced Sensitivity for the Detection of Trace Gases Using Multiple Line Integrated Absorption Spectroscopy,” Applied Optics, vol. 48, p. 5061-5066, 2009 (“Rao 2”), which is incorporated herein by reference in its entirety. This advantage comes from the fact that many laser-based spectroscopic techniques require the sample to be at a reduced pressure in order to resolve a specific absorption line, and use the line's amplitude to detect the species' concentration. When a sample is at atmospheric pressure, however, most of the additional absorption manifests itself in the broadening of the lines—not via a large increase in the line's amplitude. Thus, when dealing with broadened lines, a more accurate measure of the absorption intensity can be achieved by integrating over the absorption line as disclosed by the Hollas article. If the lines are closely spaced such that the observed spectra are the result of many overlapping lines, the direct calculation of the concentration may not be practical. Instead an experimental parameter S_{T} is defined which is equal to the number density N multiplied by the sum of the areas under the different absorption peaks (this is referred to as the total absorption signal):

S_{T}=Σᵢ∫σᵢ(v)NLdv (6)

Here, σᵢ(v) is the absorption cross-section of the i^{th} transition of the target species, and the summation is over all transitions within the selected tuning range of the laser. Using pre-calibrated reference mixtures of the desired gas, an S_{T} vs. concentration curve that characterizes a particular experimental apparatus can be defined. The unknown concentrations of the species are defined by recording its S_{T} and identifying its location on this chart. See the Rao 2 article.

### SUMMARY OF THE INVENTION

The invention is a highly sensitive trace gas sensor based on a simplified design that is capable of high precision measurements in 10's of milliseconds, i.e., real time. The sensor makes use of a Fabry-Perot (FP) semiconductor laser to conduct cavity enhanced spectroscopy (CEAS). In CEAS, the laser is coupled to a high-finesse optical cavity to obtain a long effective path length and thus high sensitivity. The multi-mode FP semiconductor laser has a broad frequency range that spans a large number of absorption lines, thereby removing the need for a single frequency, tunable laser source (as is typically used in CEAS). Additionally, the broad frequency range of the laser excites a large number of cavity modes simultaneously, thereby reducing the sensor's susceptibility to vibration. Multiple line integrated absorption spectroscopy (where one integrates the absorption spectra over a large number of rovibronic or rovibrational transitions of the molecular species) further improves the sensitivity of detection. This is accomplished by integrating the absorption spectra over a selected frequency range of the laser. Relatively high laser power is used to compensate for the low coupling efficiency of a broad linewidth laser to the optical cavity. The sensor may be used to detect any of a large number of different gases using a FP semiconductor laser with a wavelength that matches the absorption bands of target molecular species.

The present invention calls for the use of a broadband semiconductor laser with Multiple line integrated absorption spectroscopy (MLIAS), which allows the integration of the absorption signal of all of the spectral lines within the laser's frequency range simultaneously, without tuning. Thus, using a multi-mode semiconductor laser for MLIAS coupled with Cavity enhanced absorption spectroscopy (CEAS) allows one to record and average data much more quickly than with other laser-based cavity enhanced techniques (which require the laser source to be tuned), or IBB-CEAS (which requires a spectrometer to maintain selectivity). This offers the potential for highly sensitive, real-time monitoring of trace gas concentrations.

The use of a broad band laser excites a large number of cavity modes simultaneously, thereby reducing the sensor's susceptibility to vibration. In many implementations of CEAS (see the Paul article), off-axis alignment is used to excite a large number of cavity modes. Off-axis alignment is used to create a condition where the effective FSR of the cavity is significantly narrower than the laser linewidth. As a result, the laser will always be resonant with some set of cavity modes (regardless of slight changes to the cavity length due to vibrations or small amounts of drift in the laser frequency). A key design issue, however is that the cavity mirrors need to be large enough to allow multiple reflections within the cavity without causing beam overlap on the mirrors. This requirement for using large mirrors (diameter ˜50 mm) causes a complication. Specifically, many applications require the use of a low-volume cell. In order to maintain a small volume while using larger mirrors, one must reduce the spacing between the mirrors, resulting in reduced sensitivity due to the shorter path length. In place of off-axis alignment, this invention uses a laser source whose broad frequency range covers on the order of a thousand FSR (assuming a typical cavity with FSR ˜300 MHz), and thus excites a large number of cavity modes. As a result, any slight change to the cavity length due to vibrations will simply shift this array of cavity resonances to other wavelengths in which the laser is emitting (i.e., the laser will always be resonant with the cavity). As a result with this invention, there is no restriction on the cavity alignment necessary to prevent the overlapping of reflected beams (i.e., the cavity's natural FSR (c/nL) because a single pass through the cell determines the sensor performance as opposed to an effective FSR resulting from off-axis geometry), resulting in simplified alignment and improved signal-to-noise ratio.

Relatively high laser power is used to compensate for the low coupling efficiency of a broad line-width laser to the optical cavity. In an illustrative embodiment a 407 nm diode laser is used to detect trace quantities of NO₂ in Zero Air. Sensitivities of 750 ppt, 110 ppt and 65 ppt are achieved using integration times of 50 ms, 5 sec. and 20 sec., respectively.

### BRIEF DESCRIPTION OF THE DRAWINGS

The foregoing and other features of the present invention will be more readily apparent from the following detailed description and drawings of an illustrative embodiment of the invention in which:

FIG. 1 is a schematic diagram of apparatus for carrying out the demonstration of the present invention;

FIG. 2 illustrates the spectrum of a multi-mode semiconductor laser beam used in an embodiment of the present invention;

FIG. 3 shows the NO₂ absorption at 298.5 K and atmospheric pressure over the laser's wavelength range;

FIG. 4 is a plot of a CEAS absorption signal vs concentration for a 5 second integration time;

FIG. 5A illustrates a CEAS signal recorded for a period of 10 minutes using an integration time of 20 sec with Zero Air flowing through the cell at 1 liter/min, FIG. 5B uses an integration time of 5 sec, and FIG. 5C uses an integration time of 50 ms;

FIG. 6 is a log-log plot of standard deviation of a CEAS signal vs sample averaging time; and

FIG. 7 is a graph of deviation from a baseline signal during a long term stability test.

### DETAILED DESCRIPTION OF AN EXEMPLARY EMBODIMENT OF THE INVENTION

A new trace gas detection technique and its applications are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiment illustrated by the figures or description below. More specifically, some of the details provided below include a demonstration of the invention to detect NO₂. The details specific to NO₂ detection (for example the use of a multi-mode diode laser emitting near 405 nm), pertain to this demonstration and are not intended to limit the invention to this specific laser, wavelength or molecular species.

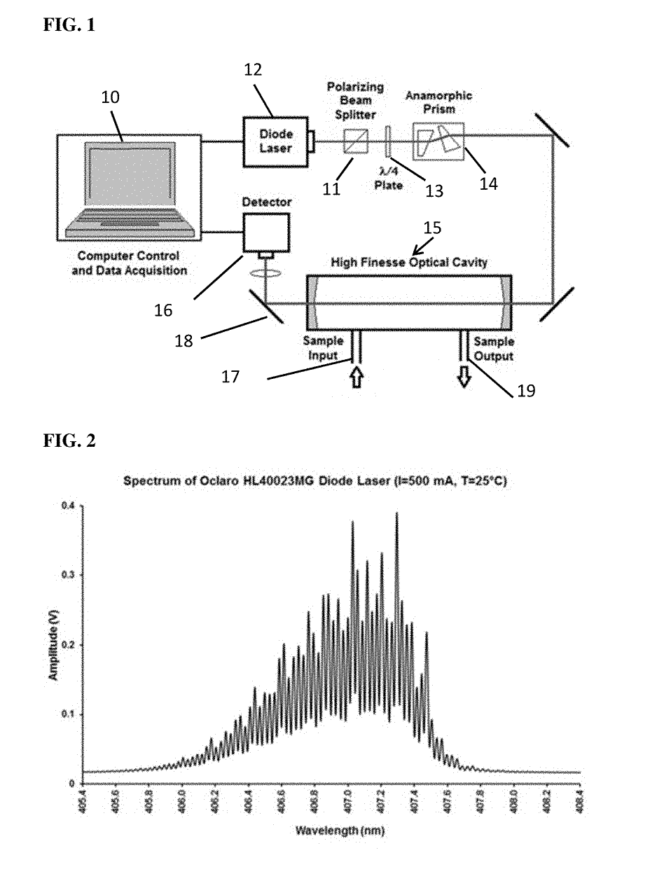

FIG. 1 shows apparatus as configured for demonstrating Cavity Enhanced Absorption Spectroscopy (CEAS) using a multi-mode diode laser by measuring trace concentrations of NO₂. The apparatus includes a diode laser 12 whose operation is directed by a computer control and data acquisition system 10. The beam from laser 12 passes through optics which include a polarizing beam splitter 11 and a quarter wave plate 13 that provide optical isolation from the back reflection of the optical cavity. The optical elements also include an anamorphic prism 14 that is used to shape the asymmetric diode laser beam.

The beam from the prism 14 is directed by mirrors so it enters a High Finesse Optical Cavity 15. In the cavity it encounters the sample gas which flows through the cavity from a gas sample input 17 to a gas sample output 19. The optical output of the cavity is reflected by a mirror through focusing optics 18 to a detector 16. Detector 16 converts the optical signal into an electrical signal that is input to the data acquisition portion of computer 10.

There are two main factors that needed to be considered for the selection of a spectral region for investigation: 1) A region with strong absorption lines; and 2) A region free from interference due to other species in the atmosphere (especially water vapor and other gases). Some of the strongest NO₂ rovibronic transitions are in the region accessible using 405 nm diode lasers. S. Voigt, J. Orphal and J. P. Burrows, “The temperature and pressure dependence of the absorption cross-sections of NO₂ in the 250-800 nm region measured by Fourier-transform spectroscopy,” J. Photochem. Photobiol. A: Chem., vol. 149, pp. 1-7, 2002 (“Voigt”), which is incorporated herein by reference in its entirety. A review of the spectra of the main atmospheric components, shows that there are no interfering species within 5 nm on either side of the laser line at 405 nm. See L. S. Rothman et al., “The HITRAN 2008 molecular spectroscopic database,” J. Quant. Spectrosc. Radiat. Transfer, vol. 110, pp. 533-572, 2009 (“Rothman”); C. N. Mikhailenko, Y. L. Babikov and V. F. Golovko, “Information-calculating system Spectroscopy of Atmospheric Gases. The structure and main functions.,” Atmos. Oceanic Opt., vol. 18, pp. 685-695, 2005 (“Mikhailenko”); and NASA, “Atmosphere, Earth Fact Sheet—Terrestrial,” [Online]. Available at: http://nssdc.gsfc.nasa.gov/planetary/factsheet/earthfact.html (“NASA”), all of which are incorporated herein by reference in their entirety.

In an embodiment of the apparatus of FIG. 1, the laser source is a high-power violet diode laser operated in CW mode with an injection current of 500 mA at a temperature of 25° C. A clean current source is used to drive the laser; a temperature controller is used to drive a thermo-electric cooler to maintain a stable, constant temperature (ΔT˜0.01° C.). The spectrum of the laser's multi-mode output is recorded using a monochromator, and contains approximately 50 modes in a Gaussian-like envelope centered at 407 nm (see FIG. 2). The mode distribution had a FWHM of approximately 1 nm and each individual mode had a FWHM of approximately 0.01 nm (˜15 GHz)

The experimental cell is a high-finesse optical cavity that is 50 cm long, and has mirrors with a reflectivity of ˜99.98% at 405 nm and a radius of curvature of 1 meter. It is important to note that the invention may use mirrors with other similar reflectivities and different radii of curvature (the ones used for the demonstration were selected in part due to the fact that they were commercially available at that time). The free spectral range (FSR) of the cavity was 300 MHz, and its resonance width was approximately 10 kHz.

Due to the cavity parameters described above and the broad frequency range of the laser source, a very small fraction of the incident laser light is coupled to the cavity. As discussed above, this is because the laser's power is spread across multiple modes, where each individual mode may be many GHz wide (in the case of the present embodiment, there are fifty modes, corresponding to a frequency range of approximately 1500 GHz, which is hundreds of FSR). The low coupling efficiency to the optical cavity is therefore primarily due to the low transmission of light at frequencies between the cavity resonances. To compensate for the low coupling efficiency, the invention employs a laser that emits at a relatively high output power (˜400 mW). Despite this high power, the manufacturer indicates that the laser is expected to have a long life (lifetime >10,000 hours), and it has been observed to have an output spectrum that is both repeatable and stable.

The cell or cavity 15 of FIG. 1 has input and output valves 17, 19 allowing test gas mixtures to flow through it at a constant rate. It is important to note that the choice of a silicon photodiode was due to its suitability for detecting light at the wavelength used in the demonstration using NO₂. When a laser of significantly different wavelength is used in the invention to detect a different gas, a different low noise detector would be used. The detector 16 output is fed to a commercial data acquisition (DAQ) interface for analysis in a computer (e.g., a laptop) 10. The signal analysis is conducted using software created using LabView for Windows. To demonstrate real-time data acquisition, data was recorded using three different integration times: 20 s, 5 s and 50 ms.

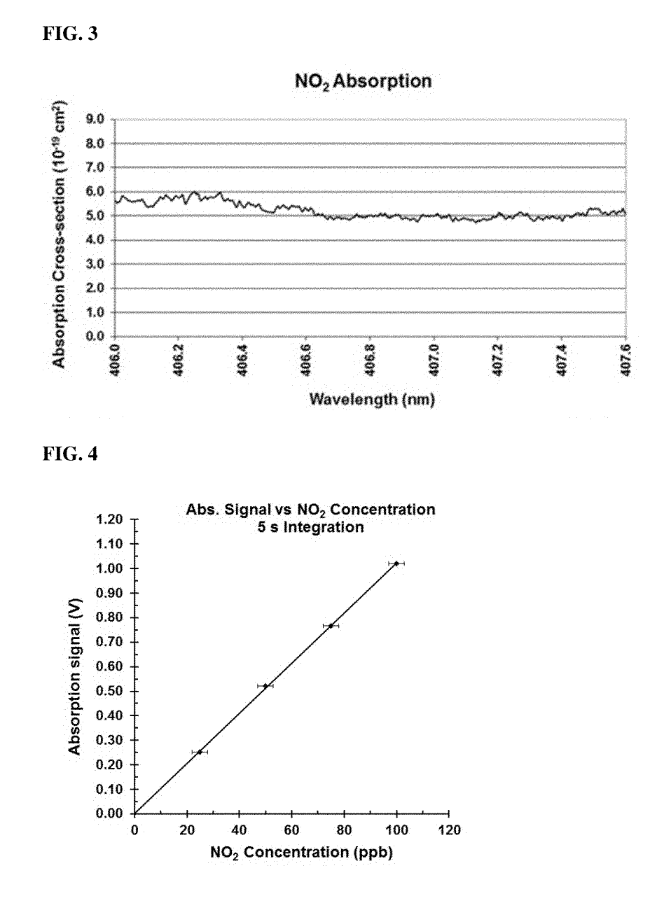

The NO₂ concentration is determined using Beer's Law, see Eq. (3) and Eq. (4). In doing so, I (v) is chosen to be the CEAS signal when only Zero Air is flowing through the cell at 1 liter/min. As a result, this signal contained loss and noise contributions from all components of the setup, and provided a baseline signal for the absorption measurements. The absorption cross-section could be treated as having a constant value of ˜5×10⁻¹⁹ cm² over the laser's wavelength range for the following reasons: 1) The close spacing of the energy levels in NO₂ and the large width of the absorption features at one atmosphere result in very broad, overlapping absorption features; (See FIG. 3); 2) Spectra recorded for the laser over the course of several hours using a monochromator shows no noticeable drift when compared with the broad absorption features; 3) The ambient (i.e., sample cell and gas) temperature is constant during runs of the equipment. A. C. Vandaele, C. Hermans, P. C. Simon, M. Carleer, R. Colins, S. Fally, M. F. Mérienne, A. Jenouvrier and B. Coquart, “Measurements of the NO₂ absorption cross-sections from 42000 cm-1 to 10000 cm-1 (238-1000 nm) at 220 K and 294 K,” J. Quant. Spectrosc. Radiat. Transfer, vol. 59, pp. 171-184, 1998 (“Vandaele”), which is incorporated herein by reference in its entirety.

The apparatus of FIG. 1 was used to detect several concentrations of NO₂ (25, 50, 75 and 100 ppb), using CEAS and a multi-mode diode laser. To demonstrate the real-time measurement capabilities, data sets were recorded using integration times of 50 milliseconds, 5 seconds and 20 seconds. The absorption signal [I(v)−I′(v)] was plotted as a function of known NO₂ concentration. FIG. 4 shows a plot of the absorption signal vs. concentration for a 5 sec. integration time, as well as a weighted linear least-squares fit of this data. The horizontal error bars represent the uncertainty in the gas mixing apparatus (±3 ppb).

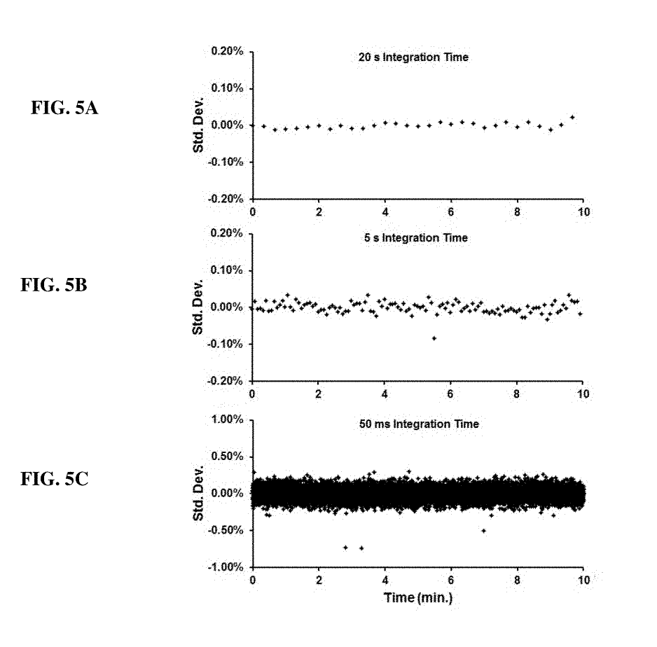

The instrument's sensitivity was calculated by determining the noise level in the CEAS signal. This was accomplished by flowing Zero Air through the cell at 1 liter/min, and recording data for 10 minutes (see FIG. 5A). The standard deviation of the CEAS signal with a 20 second integration time was found to be 0.0076%. The minimum detectable concentration (at the 1σ level) is found by dividing the voltage level of the standard deviation (0.68 mV) by the slope of the weighted linear least-squares fit of the data recorded from the NO₂ concentrations (10.2 mV/ppb). The slope is used since it incorporates uncertainties from all aspects of the measurements (e.g., repeatability of the measurement with different concentrations of NO₂). Using this data it was determined that the sensitivity of the apparatus using a 20 second integration time was approximately 65 ppt. Following the same procedure, the standard deviation for CEAS using a 5 sec. integration time was found to be 0.013% (1.12 mV), and the sensitivity was determined to be 110 ppt. Using a 50 ms integration time the standard deviation was found to be 0.079% (7.03 mV), and the sensitivity was determined to be 750 ppt. This result is comparable to both the sampling time and sensitivity achieved by Courtillot, using optical-feedback CEAS. The results in this demonstration, however, were obtained using a design that is significantly less complicated and less expensive than that of Courtillot.

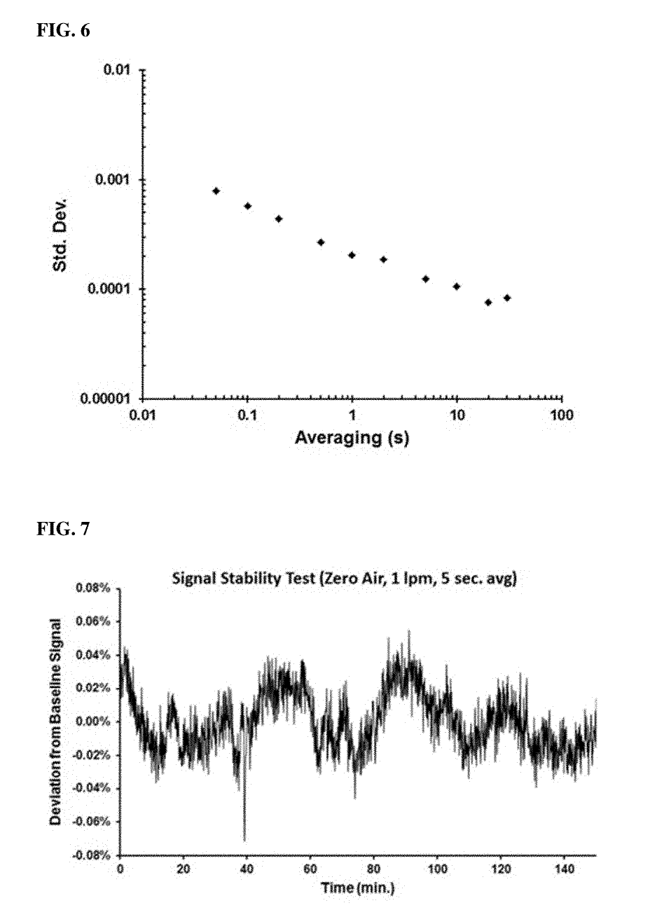

To analyze the stability of the sensor, the signal was recorded using several different averaging settings (30, 20, 10, 5, 2, 1, 0.5, 0.2, 0.1, and 0.05 sec), with 1 liter per minute of Zero Air flowing through the cell. Data was recorded for ten minutes for each setting, and the standard deviation was calculated. A log-log plot of the standard deviation vs. avg. time (FIG. 6) shows that the optimal short-term sensitivity occurs with 20 second averaging.

The present invention thus is a highly sensitive, real-time trace gas sensor using a multi-mode semiconductor laser and MLIAS coupled with cavity enhanced absorption spectroscopy. The relatively broad frequency spread of this type of laser (on the order of 1500 GHz, or 1 nm) spans a large number of absorption lines, thereby removing the need for a tunable laser source. Its frequency spread, however, is still narrow enough to maintain the specificity necessary for trace gas detection without the need for a spectrometer. CEAS enhances the sensitivity of detection by providing a path length on the order of 1 km in a small-volume cell. The broad-band source excites a large number of cavity modes, thereby minimizing effects of vibration on the signal from the optical cavity. The use of MLIAS further enhances the sensor's sensitivity and is well suited for measurements at atmospheric pressure. Though the use of a relatively broadband source results in a low coupling efficiency of the laser source to the cavity, it is addressed simply by the use of a readily available, high power semiconductor laser.

The technique demonstrated via the construction of a sensor to detect trace quantities of NO₂ in Zero Air, and sensitivities of 65 ppt, 110 ppt and 750 ppt were achieved using integration times of 20 sec., 5 sec., and 50 ms. These results are comparable to some of the most sensitive results reported. See the Fuchs and Courtillot articles as well as G. N. Rao and A. Karpf, “Extremely sensitive detection of NO2 employing off-axis integrated cavity output spectroscopy coupled with multiple-line integrated absorption spectroscopy,” Appl. Opt., vol. 50, pp. 1915-1924, 2011(“Rao 4”), which is incorporated herein by reference in its entirety. Nevertheless, the present invention makes use of a design that is simpler and significantly less expensive than other reported devices. Although the illustrated embodiment uses a 407 nm multi-mode diode laser and NO₂, the invention could be carried out using different Fabry-Perot diode lasers or Fabry-Perot quantum cascade lasers to detect other species.

While the invention has been particularly shown and described with reference to preferred embodiments thereof, it will be understood by those skilled in the art that various changes in form and details may be made therein without departing from the spirit and scope of the invention.

We claim:

1. A method for detecting trace gases in a gas sample using cavity enhanced absorption spectroscopy, comprising the steps of:
generating a continuous multi-mode laser beam with a Fabry-Perot semiconductor laser;
passing said laser beam into a high finesse optical cavity cell in which the sample gas is located, whereby the laser beam bounces back and forth in the cavity cell a number of times and exits the cavity cell; and
detecting integrated absorption in the laser beam exiting the cavity cell due to rovibronic and/or rovibrational transitions of the molecular species as it interacts with the laser beam bouncing in the cavity.

2. The method of claim 1 wherein the laser beam is of relatively high power to compensate for low cavity throughput.

3. The method of claim 1 wherein the broadband multi-mode laser beam excites a large number of cavity modes, thereby making the apparatus insensitive to vibration.

4. Apparatus for detecting trace gas species in a gas sample using cavity enhanced absorption spectroscopy, comprising:
a multi-mode semiconductor laser source that provides a continuous laser beam with a broad frequency bandwidth;
a high finesse optical cavity cell in which the sample gas is located, said cell having high reflectivity mirrors at the wavelength corresponding to the absorption features of the trace species; and
a detector for detecting the laser beam after it exits the cell

5. The apparatus of claim 4 wherein the laser is a Fabry-Perot semiconductor laser.

6. The apparatus of claim 4 wherein the cell has an entrance through which the sample gas enters the cell at one end, and an exit from which the sample gas exits at the other end.

7. The apparatus of claim 4 wherein the laser beam is of relatively high power.
